(19) United States
(12) Patent Application Publication
(10) Pub. No.: US 2014/0302178 A1
(43) Pub. Date: Oct. 9, 2014

(54) Natural Product for Decreasing Symptoms During Viral Infections

(71) Applicant: Hanan Polansky, Rochester, NY (US)

(72) Inventor: Hanan Polansky, Rochester, NY (US)

(21) Appl. No.: 13/856,592

(22) Filed: Apr. 4, 2013

(51) Int. Cl. A61K 36/82; A61K 33/00; A61K 36/54; A61K 31/352; A61K 36/484

(52) U.S. Cl. CPC A61K 36/82; A61K 31/352; A61K 36/484; A61K 36/54; A61K 33/00
USPC 424/702; 424/729; 514/456; 424/757; 424/739

(57) ABSTRACT

Gene-Eden-VIR is a natural product that decreases the severity, duration, and frequency of symptoms during a viral infection, including infections with the Human Papillomavirus (HPV), Epstein Barr Virus (EBV), Herpes Simplex Virus (HSV), Human Cytomegalovirus (HCMV), and Hepatitis C Virus (HCV).

This non-provisional application claims the benefits of the following provisional patent applications:
1. No. 61/620,997, filed on 6 Apr. 2012, entitled: Natural Product for Decreasing Symptoms During Viral Infections;
2. No. 61/620,998, filed on 6 Apr. 2012, entitled: Natural Product for Increasing Physical Strength;
3. No. 61/646,910, filed on 15 May 2012, entitled: Natural Product for Increasing Levels of Energy.

### BACKGROUND OF THE INVENTION

Current treatments for decreasing symptoms during viral infection have many limitations. Consider the following examples.

Human Papillomavirus (HPV): There are no drugs approved against the HPV (Stanley 2012). There are only procedures, such as cryotherapy, conization, and the Loop Electrosurgical Excision Procedure (LEEP) that surgically remove the abnormal growths.

Epstein Barr Virus (EBV): A few antiviral drugs inhibit EBV replication in cell culture, including the acyclic nucleoside analogues aciclovir, ganciclovir, penciclovir, and their respective prodrugs valaciclovir, valganciclovir and famciclovir, the acyclic nucleotide analogues cidofovir and adefovir, and the pyrophosphate analogue foscarnet. However, clinical studies showed that these drugs are mostly ineffective in humans (Gershburg 2005).

Herpes Simplex Virus (HSV): Two categories of antiviral treatments are available for HSV: topical and oral. The treatments include penciclovir, acyclovir, famciclovir, and valaciclovir. However, their effectiveness is limited. For instance, a meta-analysis of five placebo-controlled and two dose-comparison studies that evaluated the effect of aciclovir, famciclovir or valaciclovir on symptoms duration showed that oral antiviral therapy decreases outbreak duration and the associated pain by 1 day only (Jensen 2004).

Human Cytomegalovirus (HCMV): Drugs approved for the treatment of HCMV infections in immunocompromised individuals include ganciclovir, its oral prodrug valganciclovir, cidofovir, foscavir and fomivirsen. However, the use of these drugs in immunocompetent individuals is limited by their toxicity, poor oral bioavailability, modest efficacy, and the development of drug resistance (Andrei 2008).

Hepatitis C Virus (HCV): Therapy can cure a chronic HCV infection. A cure is measured by the “sustained virological response” (SVR), defined as undetectable HCV RNA in peripheral blood. The standard treatment for chronic HCV infections is the combination of a pegylated interferon (IFN)-α and ribavirin. This combination is effective in about 80% of the infections with the HCV genotypes 2 or 3, but only 40%-50% in infections with the genotypes 1 or 4. Lately, two new drugs were approved, telaprevir and boceprevir, with better results. However, the combinations of pegylated interferon (IFN)-α and ribavirin and telaprevir or boceprevir are associated with additional side effects, increased costs, and more complex treatment strategies (Pawlotsky 2012).

There are also some dietary supplements, which claim to decrease symptoms during viral infections. However, they offer little to no clinical proof to support such claims. In recent years, there has been an increase in the demand for natural medicine, and there is growing evidence that they are both effective and safe (Lakhan and Vieira 2010). The 2007 National Health Interview Survey (NHIS) found that approximately 40 percent of adult Americans used Complementary and Alternative (CAM) medicine in 2007 (Barnes 2007). Another recent study showed that CAM therapies are increasingly integrated into health care practices. According to the American Hospital Association's Annual Survey of Hospitals, the percentage of hospitals offering natural therapies has increased from 7.9 percent in 1998 to 19.8 percent in 2006 (Johnson 2012). However, unlike conventional medicines, natural remedies are not considered as drugs, and therefore, are not regulated as such by the FDA (FDA 2004). As a result, most natural products available today lack clinical proof for their efficacy, especially, the kind required by the FDA for new drug approval.

### BRIEF SUMMARY OF THE INVENTION

Gene-Eden-VIR is a natural product that decreases the severity, duration, and frequency of symptoms during viral infections, including infections with the Human Papillomavirus (HPV), Epstein Barr Virus (EBV), Herpes Simplex Virus (HSV), Human Cytomegalovirus (HCMV), and Hepatitis C Virus (HCV).

### DETAILED DESCRIPTION OF THE INVENTION

Gene-Eden-VIR is a natural product designed to control the viral copy number in latently infected individuals, that is, asymptomatic individuals.

Gene-Eden-VIR was formulated by analyzing the text in thousands of scientific papers with a proprietary psycholinguistic-based, data-mining program, called Computer Intuition. The research objective was to identify natural ingredients that have a strong antiviral effect against the most common viruses. In this process, the computer analyzed more than 50,000 papers. At the end of this stage, the computer assisted in selecting five ingredients: Green Tea Extract, Quercetin, Licorice Extract, Cinnamomum Extract, and Selenium.

A manual search on these five ingredients supported the Computer Intuition analysis. The search identified a few studies that directly measured the antiviral effect of these ingredients. For instance, some studies showed that catechins, found in green tea, are effective against viruses such as Epstein-Barr Virus (EBV), Herpes Simplex Virus (HSV), Hepatitis Virus B (HVB), and other viruses (Singal 2005, Lyu 2005, Chang 2003, Lin 2000). Other studies showed that quercetin inhibits EBV-EA activation in latently infected cells (Iwase 2001, Ozcelik 2006, Arena 2008). Some studies showed that glycyrrhizin and glycyrrhizic acid, found in licorice, have an antiviral effect (Sekizawa 2001, Kapadia 2002, Lin 2003, Fiore 2008). A few studies showed that the active compounds in cinnamon: cinnamaldehyde, terpenoids, eugenol, and ethyl cinnamate, have a strong antiviral effect (Benencia 2000, Tragoolpua 2007, Orihara 2008). Finally, some studies reported that selenium has an antiviral effect (Jian 2003, Wojtowicz 2004, Schrauzer 2008).

After selecting the five ingredients, the developers used the Computer Intuition program again to analyze the thousands of papers published on these five ingredients. The following table lists the number of scientific papers published on each ingredient according to PubMed as of Sep. 1, 2009.

TABLE 1
Scientific papers per ingredient according to PubMed

| | Number of |
|---|---|
| Ingredient | Scientific Papers |
| Green Tea Extract | 3,413 |
| Quercetin | 6,753 |
| Licorice Extract | 2,215 |
| Cinnamomum Extract | 913 |
| Selenium | 2,004 |
| Total | 15,298 |

At the end of this stage, the developers determined the final formula of Gene-Eden-VIR: Quercetin 100 mg, Green Tea Extract 150 mg, Cinnamon Extract 50 mg, Selenium 100 mcg, and Licorice Extract 25 mg. Gene-Eden-VIR was introduced in the marketplace at the end of 2009.

The current invention centers on the fact that Gene-Eden-VIR decreases the severity, duration, and frequency of symptoms during viral infections, including infections with the Human Papillomavirus (HPV), Epstein Barr Virus (EBV), Herpes Simplex Virus (HSV), Human Cytomegalovirus (HCMV), and Hepatitis C Virus (HCV).

### Examples

The following section reports the results of a clinical study that proved the antiviral properties of Gene-Eden-VIR. Specifically, it showed that Gene-Eden-VIR decreased the severity, duration, and frequency of symptoms during infections with the Human Papillomavirus (HPV), Epstein Barr Virus (EBV), Herpes Simplex Virus (HSV), Human Cytomegalovirus (HCMV), and Hepatitis C Virus (HCV).

### Methods

### Treatment

Participants used 1, 2, 3, or 4 capsules of Gene-Eden-VIR per day. The duration of treatment ranged from 2 to 54 weeks.

### Questionnaire

We used a self-developed questionnaire called the Natural Origin Treatment Clinical Questionnaire (NotCiq). The following section presents the questionnaire.

The NotCiq questionnaire is a patient reported outcome (PRO) instrument. The purpose of a PRO instrument is to capture the patient's experience. It should be a reliable measure that can support the claimed concept (FDA, 2006). As such, the primary endpoint should be defined. Our main endpoint was symptoms associated with viral infections. Meaning, the objective of the study was to measure the effect of the treatment with Gene-Eden-VIR on the symptoms of the viral infection as they are reported by the treated participants.

To develop the questionnaire, several questionnaires found in the literature that measured similar constructs were analyzed. The analysis considered the purpose of study, the research question, the scale and response format, the phrasing of tested items, and the process of determining its reliability and validity. At the end, a five section questionnaire was created. One section was designed to measure the changes in general health. A second section centered on the changes in the severity, duration, and frequency of the symptoms during a viral infection. A third section centered on changes in the ability to perform physical tasks, a fourth on energy/fatigue, and a fifth on the ability to perform mental tasks. The general health section included 5 questions. The section explored areas such as current disease and the change in general health. The symptoms section included 5 questions, two questions on severity, two questions on duration, and one question on the frequency of symptoms. The physical section included 5 questions, the energy/fatigue section included 5 questions, and the mental section included 6 questions. NotCiq included both open and closed-ended questions. The answers to the closed-ended questions were on a scale of 1 to 7, where “1” corresponded to “Poor health,” “Very Severe,” “Extremely Interfered,” “All the time,” “No relief,” or “Frequently appeared,” and 7 to “Unnoticeable,” “Not At All,” “Constant Relief,” or “Never Appeared,” etc.

The study collected the answers to the NotCiq instrument by phone interviews. The study used two independent companies that specialized in outbound call services for performing the interviewers, one company from the US and one from Israel. The interviewers were blinded to the objective of the study. All interviews were recorded.

The instrument was pre-tested on a small sample of Gene-Eden-VIR users to evaluate both the sensitivity and clarity of the questions.

### Population

We randomly selected participants from the Gene-Eden VIR customer database that includes all Gene-Eden-VIR current and past customers. The traditional response rate to phone interviews is 10-15%. Since the study was aiming to collect 100-150 interviews, the study used a computerized system to randomly create a call list of a 1000 customers. Out of these customers, 100 agreed to participate. From these participants, the study excluded customers who were using Gene-Eden-VIR for other purposes, such as treatment for cancer, chronic diseases, hypertension, etc. The final list of participants included 60 Americans, ages 20 to 66, infected with the following viruses: the Human Papillomavirus (HPV), Epstein Barr Virus (EBV), Herpes Simplex Virus (HSV), Human Cytomegalovirus (HCMV), and the Hepatitis C Virus (HCV). The diagnosis was done by the participant's physician. Each participant reported as having specific symptoms including, for HPV participants reported genital warts, low and high grade cervical dysplasia, abnormal Pap smear results, and general symptoms, including, blisters, cold sores, hives, skin tabs, panic attacks, depression, kidney problems, sleeping problems, liver problems, fever, fatigue, sore throat, swollen lymph nodes, diarrhea, and weight loss. Since the objective of the study was to test the effect of Gene-Eden-VIR on symptoms associated with viral infections, the study excluded participants who reported no symptoms. That is, participants who reported a 7 point score on the pre-treatment question were excluded. Such a score indicates that the participant does not suffer from the symptom represented by the question regardless of the treatment the participant actually received. This exclusion still preserves the intention to treat (ITT) principle.

We considered participants who stopped taking Gene-Eden-VIR for a month or more before data collection as past users. All other participants were considered as present users.

### Controls

The Gene-Eden-VIR post marketing study includes a pre-treatment concurrent control and an historical control. To create an historical control, the study divided the original test group into two subgroups, present users and past users. Generally, an historical control is a separate group. However, since the study did not have a separate group of non-users, it used the past users as a proxy for an historical control.

### Statistical Analysis

We tested the statistical difference between the score of ‘pre-treatment’, which is the numeric answer each participant used to describe his symptoms before the treatment started, to the score of ‘post-treatment’, which is the numeric answer each participant used to describe his symptoms after the treatment was completed. The delta (a), that is, the difference in scores between the answers to the pre-treatment and post-treatment question was also calculated. Then, the statistical difference between the deltas was tested. These tests were performed in a then-test model for both present and past users.

Statistical analysis was performed using a two-sample assuming unequal variances t-test.

The research defined the primary endpoint as a statistically significant increase in the score from pre-treatment to post-treatment on the raw answers and on the deltas.

### Results

The participants reported no side effects from Gene-Eden-VIR.

Out of the 60 infected participants, 7 reported perfect general health and no symptoms (that is, a score 7 out of 7 on the pre-treatment questions on both the general health question and the symptoms questions), and 7 participants reported perfect general health with some symptoms. Fifty four percent (25/46) of the individuals that reported less then perfect health reported an improvement in general health.

Out of the 60 infected participants, 41 reported having some symptoms. Seventy three percent (30/41) of the individuals treated with Gene-Eden-VIR reported a decrease in their symptoms. Specifically, they reported a decrease in the severity of their symptoms (p=0.006, n=45), a decrease in the duration of their symptoms (p=0.009, n=34), and a decrease in the frequency of their symptoms (p<0.001, n=31) (Table 2).

These clinical results show that individuals infected with the following viruses: the HPV, EBV, HCMV, HSV or HCV report a safe decrease in their symptoms following treatment with Gene-Eden-VIR.

Note that although 73% of the participants with symptoms reported a decrease in their symptoms, only 54% reported an improvement in their general health. These numbers suggest that some participants do not perceive a decrease in symptoms associated with viral infections as an improvement in general health. In their mind, viral symptoms and general health are somewhat disconnected.

TABLE 2
Pre-treatment vs. post-treatment symptoms as reported by the participants

| | | Pre- | Post- | |
|---|---|---|---|---|
| | No. of | treatment | treatment | |
| Question | Participants* | score | score | P Value |
| A5 - General health | 46 | 4.67 | 5.59 | <0.001 |
| B1-B2 - Severity of | 45 | 4.90 | 5.49 | 0.006 |
| Symptoms | | | | |
| B3-B4 - Duration of | 34 | 4.79 | 5.46 | 0.009 |
| Symptoms | | | | |
| B5 - Frequency of | 31 | 2.42 | 5.23 | <0.001 |
| Symptoms | | | | |

*The statistical analysis on the Severity of Symptoms, and on the Duration of Symptoms, was conducted using one score per participant. The score was equal to the average answers of the participant to questions B1 and B2, and B3 and B4, respectively.

Following treatment with Gene-Eden-VIR, the participants also reported an increase in their ability to perform physical tasks (questions C1-C6, p<0.001, n=47), an increase in their energy levels (or decrease in their fatigue) (questions D1-D5, p<0.001, n=54), and an increase in their abilities to perform mental tasks (questions E1-E6, p=0.042, n=44, Table 3).

TABLE 3
Pre-treatment vs. post-treatment physical, energy/fatigue, and mental

| | | Pre- | Post- | |
|---|---|---|---|---|
| | No. of | treatment | treatment | |
| Question | Participants* | score | score | P Value |
| Physical | 47 | 4.90 | 5.55 | <0.001 |
| Energy/Fatigue | 54 | 4.57 | 5.35 | <0.001 |
| Mental | 44 | 5.03 | 5.42 | 0.042 |

*Statistical analysis was performed using a single score for each participant. The score was equal to the average of all answers in the relevant block of questions, see questionnaire, section C, D, and E.

We could not test for a dose effect since the number of participants who took 1, 3 or 4 capsules per day was too small for statistical analysis.

To test for a duration effect, the study compared the change (Δ) from pre-treatment to post-treatment in participants who took Gene-Eden-VIR for less then 2 month and those who took Gene-Eden-VIR for 2 months or more. The results showed that participants who took Gene-Eden-VIR for the longer period reported a 220% larger decrease in their symptoms (p=0.044, n=32, Table 4).

TABLE 4
Duration of treatment: less then 2 month vs. 2 months or more

| | | Change (Δ) from | |
|---|---|---|---|
| Duration of | No. of | pre-treatment to | |
| treatment | Participants* | post-treatment | Statistics |
| Less then 2 months | 13 | 0.43 | p = 0.044, n = 32 |
| 2 months or more | 19 | 0.95 | |

*Statistical analysis was performed using the change in scores from pre-treatment to post-treatment reported by present users only. The analysis used one score per participant. The score was equal to the average of the answers to questions B1-B5.

To test for a possible interviewer bias, the study compared the answers to the pre-treatment questions collected by the American and the Israeli call centers. The study also compared the answers to the post-treatment questions between the two call centers. In both cases, the difference between the answers was statistically insignificant (p=0.30, n=154, for pre-treatment, and p=0.36, n=154, for post-treatment, Table 5). This means that although the centers included different interviewers from different cultures working at different times of day, Americans working during the day and Israelis working during the night, the answers were similar. Hence, the results showed no interviewer bias. Similar results were obtained for the other sections of the questionnaire (physical, energy/fatigue, mental, data not shown).

TABLE 5
Answers collected by USA vs. Israel call centers

| | USA | | Israel | | |
|---|---|---|---|---|---|
| | No. of | | No. of | | |
| Variable | Answers* | Score | Answers | Score | Statistics |
| Pre-treatment | 81 | 3.03 | 73 | 2.74 | p = 0.30, n = 154 |
| Post-treatment | 81 | 5.04 | 73 | 5.36 | p = 0.36, n = 154 |

*The data may include up to five answers per participant, see questionnaire, section B.

To test for a possible selection bias, the study compared the answers to the pre-treatment questions by the past and present users of Gene-Eden-VIR. The study also compared the answers to the post-treatment questions between the two groups, and the change (Δ) from pre-treatment to post-treatment. In all three tests, the difference between the answers was insignificant (p=0.18, n=154, for pre-treatment, p=0.72, n=154, for post-treatment, and p=0.46, n=154, for the change (Δ), Table 6). This means that, statistically, the answers by the present users are the same as the answers by the past users, and therefore, there is no selection bias.

TABLE 6
Answers given by past vs. present users

| | Past | | Present | | |
|---|---|---|---|---|---|
| | No. of | | No. of | | |
| Variable | Answers* | Score | Answers* | Score | Statistics |
| Pre-treatment | 46 | 3.17 | 108 | 2.77 | p = 0.18, n = 154 |
| Post-treatment | 46 | 5.28 | 108 | 5.16 | p = 0.72, n = 154 |
| Change (Δ) | 46 | 2.11 | 108 | 2.39 | p = 0.47, n = 154 |

*The data may include up to five answers per participant, see questionnaire, section B.

An issue unique to natural products is the concern about the therapeutic consistency of marketed products. See discussion on this issue in the FDA guidelines for botanical New Drug Applications (NDA). To test the therapeutic consistency of Gene-Eden-VIR, the study compared the two batches used by the participants. The capsules in these batches were produced at two different manufacturing sites, and completed about 10 months apart. The results showed that the answers given by the participants who used the capsules from Batch 1 were the same as those given by the participants who used the capsules from Batch 2 (p=0.988, n=160, Table 7). Hence, the results indicated that, although Gene-Eden-VIR is a natural product, its formula has therapeutic consistency.

TABLE 7
Answers Per Batch: Batch 1 vs. Batch 2

| | | Change (Δ) from pre- | |
|---|---|---|---|
| | No. of | treatment to post- | |
| Batch | Answers* | treatment | Statistics |
| 1 | 81 | 2.20 | p = 0.988 |
| 2 | 79 | 2.20 | n = 160 |

*Note that the data may include up to five answers per participant, see questionnaire, section B.

### SUMMARY

According to the FDA website, the “FDA uses postmarketing study commitments to gather additional information about a product's safety, efficacy, or optimal use.” This post marketing study was used for the same objective, that is, to test the efficacy, safety, and optimal use of Gene-Eden-VIR during viral infections. The study showed that that individuals infected with the Human Papillomavirus (HPV), Epstein Barr Virus (EBV), Herpes Simplex Virus (HSV), Human Cytomegalovirus (HCMV), or Hepatitis C Virus (HCV) reported a safe decrease in their symptoms following treatment with Gene-Eden-VIR. The participants also reported an increase in their ability to perform physical tasks, an increase in their level of energy (or decrease in their level of fatigue), and increase in their ability to perform mental tasks, and an improvement in their general health.

The results are consistent. The study showed a statistical significant decrease in the severity, duration, and frequency of symptoms. The results also showed a duration effect. Participants treated for three months or more reported a larger decrease in their symptoms compared to those treated for less then three months.

The results are robust. They showed no interviewer bias, no selection bias, and therapeutic consistency of the Gene-Eden-VIR formula under varying manufacturing conditions.

The results showed an interesting response shift. It seems that the participants tended to forget how bad their symptoms were before taking Gene-Eden-VIR. This was unexpected. Most studies on response shift reported an effect in the opposite direction. Participants, who experience an improvement, tend to exaggerate their initial pain and suffering in a ‘then-test’. See, for instance, Ring 2005. See also FIG. 1 in Schwartz 2006. We believe that the source of the difference between these studies and the results in our study is the condition of the participants. In this study, the participants suffered from episodes. In the other studies, the participants suffered from a stable disease. During episodes, the participants experience quick transitions between symptoms and relief. Hence, they can easily compare the two states and realize how bad their symptoms are. In a stable disease, there is no reference point. Hence, only when they later recover, they form a more accurate appreciation of the severity of their initial condition. It seems that in episodes, the patients form an accurate perception during the ‘pre-test’ time. In contrast, in a stable disease, they form an accurate perception during the ‘then-test’ time.

This post marketing clinical study does not include a placebo control, that is, it is not a double blinded study. Placebo controlled studies are the gold standard in medical research in pre marketing clinical studies. However, except in rare cases, post marketing studies do not use placebo controls. They use other controls recommended by the FDA.

According to the FDA Guidance to the Industry (FDA 2001):
“Control groups have one major purpose: to allow discrimination of patient outcomes (for example, changes in symptoms, signs, or other morbidity) caused by the test treatment from outcomes caused by other factors, such as the natural progression of the disease, observer or patient expectations, or other treatment. The control group experience tells us what would have happened to patients if they had not received the test treatment or if they had received a different treatment known to be effective.”

The FDA guidance mentions five types of controls used in both pre marketing and post marketing studies: (1) Placebo Concurrent Control, (2) Pre-treatment Concurrent Control, (3) Dose-response Concurrent Control, (4) Active (Positive) Concurrent Control, (5) External Control (Including Historical Control). The External Control “can be a group of patients treated at an earlier time (historical control).”

The Gene-Eden-VIR post marketing study includes a pre-treatment concurrent control and an historical control.

In a post marketing study one must consider the effect of cognitive dissonance, that is, the condition where people are holding two conflicting beliefs. Under cognitive dissonance, people have a motivational drive to reduce the conflict by altering these beliefs, adding new ones to create a consistent belief system, or reducing the importance of one of the conflicting elements. In this study one can expect the existence of cognitive dissonance that may result from guilt, commitment, or the need to justify the purchasing and using of the product to oneself by believing that the product actually works. It is possible that the past users are less likely to experience such dissonance since they no longer use the product, and hence, feel no commitment to report a positive effect. Moreover, past users might have the opposite dissonance, that is, a need to justify their decision to stop using the product, and hence, report no effect or even a negative effect. The results showed that past and present users report a similar decrease in their symptoms. This similarity indicates that cognitive dissonance was not involved in determining the participants' reports.

All participants who started the study completed it; therefore the study has no follow-up bias.

It should be noted that although the study tested for some biases, others are still possible, for instance, the non-responsive bias.

The results are not likely to be a placebo effect. The current predominant and well-proven theories on the placebo effect suggest that its main mechanisms are conditioned reflexes and patient expectations (Breidert and Hofbauer 2009). The Gene-Eden-VIR product literature, website, or any other written or oral public communications, did not mention the possibility of a change in symptoms, and specifically, the severity, duration, and frequency of symptoms. Hence, the participants in this study could not have been primed for, or expect the reported effects. This lack of conditioned reflexes and patient expectations minimizes the possibility of a placebo effect, and supports the possibility of a physiological effect.

This study relies on patient reported outcomes (PROs). Past studies showed that PROs had a significant role in the development and evaluation of new medicines (Willke 2004). From the years 1997 to 2002, the FDA approved 23 new drugs based on PRO endpoints only. They include six anti-migraine products (Amerge®, Axert®), several anti-epileptics (Gabitril®, Keppra®), and a variety of other therapy classes (Tamiflu®, Relenza®) (Willke 2004).

Consider the FDA's opinion on the issue. According to the FDA 2006 (underline added):
“A PRO is a measurement of any aspect of a patient's health status that comes directly from the patient (i.e., without the interpretation of the patient's responses by a physician or anyone else). In clinical trials, a PRO instrument can be used to measure the impact of an intervention on one or more aspects of patients' health status, hereafter referred to as PRO concepts, ranging from the purely symptomatic (response of a headache) to more complex concepts (e.g., ability to carry out activities of daily living), to extremely complex concepts such as quality of life, which is widely understood to be a multidomain concept with physical, psychological, and social components. Data generated by a PRO instrument can provide evidence of a treatment benefit from the patient perspective.
Generally, findings measured by PRO instruments may be used to support claims in approved product labeling if the claims are derived from adequate and well-controlled investigations that use PRO instruments that reliably and validly measure the specific concepts at issue.
2.1 Why use patient-reported outcome instruments in medical product development?
PRO instruments are included in clinical trials for new medical products because (1) some treatment effects are known only to the patient; (2) there is a desire to know the patient perspective about the effectiveness of a treatment; or (3) systematic assessment of the patient's perspective may provide valuable information that can be lost when that perspective is filtered through a clinician's evaluation of the patient's response to clinical interview questions.”

PRO are also discussed in scientific papers. According to Demuro 2012 (underline added):
“Patient-Reported Outcome (PRO) use is particularly common for products developed to treat chronic, disabling conditions where the intention is not necessarily to cure but to ameliorate symptoms, facilitate functioning, or improve quality of life. PROs are the primary end points in clinical trials evaluating drug products for disease areas such as irritable bowel syndrome, migraine, and pain. PROs provide key supportive data in many other disease areas, such as insomnia, asthma, and psychiatric disorders. In oncology, PROs are commonly used to assess both treatment benefits and toxicity to fully evaluate the impact of treatment on health-related quality of life (HRQOL). PROs can also be used in clinical trials to assess treatment satisfaction, compliance, and caregiver burden.
Willke and colleagues [2] conducted a review of drug labels to understand the use of PROs compared with other trial end points. That research identified the inclusion of PROs as efficacy end points in approximately 30% of all labels reviewed between 1997 and 2002. In 2006, the Food and Drug Administration (FDA) released a draft guidance for use of PROs in clinical trials, followed by a final guidance in 2009, Guidance for Industry: Patient Reported Outcome Measures: Use in Medical Product Development to Support Labeling Claims, providing a blueprint for the use of PROs in clinical trials. The guidance documents were intended to influence the appropriate development, validation, and use of PRO measures to facilitate a positive regulatory review in support of label claims.
Gnanasakathy and colleagues built on the work previously conducted by Willke and colleagues and reported the frequency of PROs in recently approved drug labels. Specifically, these authors found that PRO claims were granted for approximately 24% of all labels reviewed between January 2006 and December 2010.”

According to the FDA, PROs are a valid and valuable source for measuring the efficacy of new drugs. PROs are reliable enough to warrant an approval of a label claim for a new drug. Obviously, if the FDA regards this source of data as valid and valuable, so should the medical community. And indeed, many major journals publish clinical study that relies on patient reported outcomes.

Gene-Eden-VIR was formulated by analyzing thousands of scientific papers with Computer Intuition. The basic premise of the computer program is that every future event is preceded by hints, and that the key to predicting these events is recognizing the significance of these hints.

In 1996, the first author of this paper completed a prototype of a psycholinguistic-based data-mining program that analyzes scientific text and assigns a rating to all ideas found in the text. The higher the rating, the more it hints on future events.

The following is a description of one prospective application of Computer Intuition. In 1995, Frederiksen published a paper entitled: Diagnostic Imaging in Dental Implantology. At the time, Frederiksen was one of the world leading experts on the subject. To test the predictive power of the Computer Intuition analysis, Almog and Heisler from the University of Rochester devised a test. They conducted a Medline search for papers published between 1980 and 1996 using keywords relevant to the subject of diagnostics, imaging, and dental implantology. The search identified 34 papers. The content of these papers was analyzed with Computer Intuition.

The analysis produced three ideas. Two ideas were identical to the main conclusion described in Frederiksen's paper. This, by itself, was an impressive achievement. By using Computer Intuition, Almog and Heisler duplicated the results of a world leading expert quickly and inexpensively. However, while it took Frederiksen decades to build his expertise, Almog and Heisler acquired similar expertise within weeks.

The third idea suggested a new technology. This technology was not mentioned in Frederiksen's paper. The three ideas were published in 1997.

How predictive was the Computer Intuition analysis? In 2006, Almog, Frederiksen, and four colleagues analyzed the academic and commercial field of diagnostic imaging in oral implantology, and published their results. In their paper, they reported an interesting observation. Beginning in 2000, three years after the publication of the Computer Intuition research, “numerous companies from technology-transfer and commercial standpoint have introduced technology platforms that offer planning and guidance systems to facilitate dental implant placement procedures,” the same technology proposed by the third idea three years earlier. This observation confirms the Computer Intuition based prediction.

A possible limitation of this study is the subjective report of symptoms. One might argue that the participants' have evaluated the effect of Gene-Eden-VIR on symptoms, which are unrelated to their infection. To address this question, the symptoms reported by the participants were compared to the standard signs and symptoms reported in the literature (“Harrison's principles of internal medicine,” 18th edition). The comparison clearly showed that the reported symptoms and the major standard symptoms of viral infection as found in the literature overlapped (data not shown).

The size of the study group is a major concern in clinical studies. A group that is too small may fail to show a positive effect of the treatment. In addition, a small group could also misrepresent the diversity in the population. The standard principle for multivariate behavioral research is at least 10 patients at endpoint per dependent measure (Harvey and Keefe 2001). This study included one endpoint dependent measure (the change in symptoms from pre-treatment to post-treatment). This study population included 60 individuals. Hence, the size of the study group in this study is adequate.

One might also question the reliability of the participants recall due to the long duration of the time period under investigation (up to 54 weeks). This study used a ‘then-test’ method. This method, also known as the retrospective pre-test-post-test design method, asks participants at the post-test period to think back to the pre-test period and retrospectively rate their condition. The ‘response shift’ is defined as the difference between the ‘pre-test’ and the ‘then-test’ ratings. Currently, the response shift is a well documented and extensively research phenomenon (Schwartz 2006). According to the literature on ‘response shifts’, participants may alter their internal standards, values, or conceptualization of their quality of life when experiencing changes in health states. These response shifts can affect or distort the reported scores and undermine the credibility of the observed medical or psychosocial effects. Many studies reported that after participants experience an improvement in their health, a then-test tends to show a decrease in the initial assessment of the original level of well being.

Since this clinical study uses the ‘then-test’ method, the study tested for a possible response shift by comparing the answers to the pre-treatment question at one month and at three months or more. The results showed a statistically significant increase in the pre-treatment score over time (p=0.029). The results indicated that the participants experience a response shift, however, in the opposite direction from what was expected (+0.6844, p=0.029). This response shift suggests that the participant do not tend to exaggerate, but tend to forget how bad their symptoms were before taking Gene-Eden-VIR. The tendency to forget adds support to the statistical significance of the results in this study.

The developers of Gene-Eden-VIR predicted that Gene-Eden-VIR will have antiviral properties. This post marketing clinical study shows that, as predicted by the Computer Intuition analysis, individuals infected with viruses report a safe decrease in the severity, duration, and frequency of symptoms following treatment with Gene-Eden-VIR. As in the Almog and Heisler research, the results of this research confirm the Computer Intuition based prediction. The difference between the studies is that Almog and Heisler research used the results of the Computer Intuition analysis to predict the need for a new medical devise. In this research, the developers of Gene-Eden-VIR used the Computer Intuition analysis to predict the clinical results of a new formulation. Both were right.

To conclude, the developers of Gene-Eden-VIR used the results of the Computer Intuition analysis to design a product that targets latent viruses in infected individuals. This post marketing clinical study showed that infected individuals treated with the natural product Gene-Eden-VIR reported a safe decrease in their symptoms. These results prove that Computer Intuition, a psycholinguistic-based data-mining program of scientific text, can predict clinical results. Finally, the currently approved treatments for individuals infected with the HPV, EBV, HSV, HCMV, and HCV, have limited efficacy and many side effects. This study showed that the natural product Gene-Eden-VIR safely and effectively decreases symptoms in individuals infected with these viruses.

### REFERENCES

1. Stanley M A. Genital human papillomavirus infections: current and prospective therapies. J Gen Virol. 2012 April; 93(Pt 4):681-91
2. Gershburg E, Pagano J S. Epstein-Barr virus infections: prospects for treatment. J Antimicrob Chemother. 2005 August; 56(2):277-81
3. Jensen L A, Hoehns J D, Squires C L. Oral antivirals for the acute treatment of recurrent herpes labialis. Ann Pharmacother, 38 (2004), pp. 705-709
4. Andrei G, De Clercq E, Snoeck R. Novel inhibitors of human CMV. Curr Opin Investig Drugs. 2008 February; 9(2):132-45
5. Pawlotsky J M. New antiviral agents for hepatitis C. F1000 Biol Rep. 2012; 4:5
6. Lakhan S E, Vieira K F. Nutritional and herbal supplements for anxiety and anxiety-related disorders: systematic review. Nutr J. 2010 Oct. 7; 9:42
7. Barnes P M, Bloom B, Nahin R L. Complementary and alternative medicine use among adults and children: United States, 2007. Natl Health Stat Report. 2008 Dec. 10; (12):1-23
8. Johnson P J, Ward A, Knutson L, Sendelbach S. Personal use of complementary and alternative medicine (CAM) by U.S. health care workers. Health Serv Res. 2012 February; 47(1 Pt 1):211-27
9. US Department of Health and Human Services. Food and Drug Administration. Center for Drug Evaluation and Research (CDER). Guidance for industry. Botanical drug products. 2004
10. Almog D M, Heisler E M. Computer intuition: guiding scientific research in imaging and oral implantology. J Dent Res. 1997 October; 76(10):1684-9
11. Singal A, Kaur S, Tirkey N, Chopra K. Green tea extract and catechin ameliorate chronic fatigue-induced oxidative stress in mice, J Med Food. 2005 Spring; 8(1):47-52.
12. Lyu S Y, Rhim J Y, Park W B. Antiherpetic activities of flavonoids against herpes simplex virus type 1 (HSV-1) and type 2 (HSV-2) in vitro. Arch Pharm Res. 2005 November; 28(11):1293-301.
13. Chang L K, Wei T T, Chiu Y F, Tung C P, Chuang J Y, Hung S K, Li C, Liu S T. Inhibition of Epstein-Barr virus lytic cycle by (−)-epigallocatechin gallate. Biochem Biophys Res Commun. 2003 Feb. 21; 301(4):1062-8.
14. Lin L C, Kuo Y C, Chou C J. Anti-herpes simplex virus type-1 flavonoids and a new flavanone from the root of Limonium sinense. Planta Med. 2000 May; 66(4):333-6.
15. Iwase Y, Takemura Y, Ju-ichi M, Mukainaka T, Ichiishi E, Ito C, Furukawa H, Yano M, Tokuda H, Nishino H. Inhibitory effect of flavonoid derivatives on Epstein-Barr virus activation and two-stage carcinogenesis of skin tumors. Cancer Lett. 2001 Nov. 28; 173(2):105-9.
16. Ozcelik B, Orhan I, Toker G. Antiviral and antimicrobial assessment of some selected flavonoids. Z Naturforsch C. 2006 September-October; 61(9-10):632-8.
17. Arena A, Bisignano G, Pavone B, Tomaino A, Bonina F P, Saija A, Cristani M, D'Arrigo M, Trombetta D. Antiviral and immunomodulatory effect of a lyophilized extract of Capparis spinosa L. buds. Phytother Res. 2008 March; 22(3):313-7.
18. Fiore C, Eisenhut M, Krausse R, Ragazzi E, Pellati D, Armanini D, Bielenberg J Antiviral effects of Glycyrrhiza species. Phytother Res. 2008 February; 22(2):141-8
19. Kapadia G J, Azuine M A, Tokuda H, Hang E, Mukainaka T, Nishino H, Sridhar R., Inhibitory effect of herbal remedies on 12-O-tetradecanoylphorbol-13-acetate-promoted Epstein-Barr virus early antigen activation. Pharmacol Res. 2002 March; 45(3):213-20
20. Lin J C. Mechanism of action of glycyrrhizic acid in inhibition of Epstein-Barr virus replication in vitro. Antiviral Res. 2003 June; 59(1):41-7
21. Sekizawa T, Yanagi K, Itoyama Y. Glycyrrhizin increases survival of mice with herpes simplex encephalitis. Acta Virol. 2001 February; 45(1):51-4.
22. Benencia F, Courreges M C. In vitro and in vivo activity of eugenol on human herpesvirus. Phytother Res. 2000 November; 14(7):495-500.
23. Tragoolpua Y, Jatisatienr A. Anti-herpes simplex virus activities of Eugenia caryophyllus (Spreng.) Bullock & S. G. Harrison and essential oil, eugenol. Phytother Res. 2007 December; 21(12):1153-8.
24. Orihara Y, Hamamoto H, Kasuga H, Shimada T, Kawaguchi Y, Sekimizu K., A ilkworm baculovirus model for assessing the therapeutic effects of antiviral compounds: characterization and application to the isolation of antivirals from traditional medicines. J Gen Virol. 2008; 89(Pt 1):188-94.
25. Jian S W, Mei C E, Liang Y N, Li D, Chen Q L, Luo H L, Li Y Q, Cai T Y. Influence of selenium-rich rice on transformation of umbilical blood B lymphocytes by Epstein-Barr virus and Epstein-Barr virus early antigen expression. Ai Zheng. 2003; 22(1):26-9
26. Wojtowicz H, Kloc K, Maliszewska I, Mlochowski J, Pietka M, Piasecki E. Azaanalogues of ebselen (selenium-containing agents) as antimicrobial and antiviral agents: synthesis and properties. Farmaco. 2004 November; 59(11):863-8. Azaanalogues of ebselen are selenium-containing agents
27. Schrauzer G N. Effects of selenium and low levels of lead on mammary tumor development and growth in MMTV-infected female mice. Biol Trace Elem Res.
2008 December; 125(3):268-75. Epub 2008 Aug. 26.
28. U.S. Department of Health and Human Services FDA Center for Drug Evaluation and Research. Guidance for industry: patient-reported outcome measures: use in medical product development to support labeling claims: draft guidance. Health Qual Life Outcomes. 2006 Oct. 11; 4:79.
29. Ring L, Höfer S, Heuston F, Harris D, O'Boyle C A. Response shift masks the treatment impact on patient reported outcomes (PROs): the example of individual quality of life in edentulous patients. Health Qual Life Outcomes. 2005 Sep. 7; 3:55
30. Schwartz C E, Bode R, Repucci N, Becker J, Sprangers M A, Fayers P M. The clinical significance of adaptation to changing health: a meta-analysis of response shift. Qual Life Res. 2006 November; 15(9):1533-50
31. US Department of Health and Human Services. Food and Drug Administration. Center for Drug Evaluation and Research (CDER). Guidance for Industry, E 10 Choice of Control Group and Related Issues in Clinical Trials. May 2001
32. Breidert M, Hofbauer K. Placebo: Misunderstandings and Prejudices. Dtsch Arztebl Int. 2009 November; 106(46):751-5
33. Willke R J, Burke L B, Erickson P. Measuring treatment impact: a review of patient-reported outcomes and other efficacy endpoints in approved product labels. Control Clin Trials. 2004 December; 25(6):535-52
34. Demuro C, Clark M, Mordin M, Fehnel S, Copley-Merriman C, Gnanasakthy A. Reasons for Rejection of Patient-Reported Outcome Label Claims: A Compilation Based on a Review of Patient-Reported Outcome Use among New Molecular Entities and Biologic License Applications, 2006-2010. Value Health. 2012 May; 15(3):443-8
35. Frederiksen N L. Diagnostic imaging in dental implantology. Oral Surg Oral Med Oral Pathol Oral Radiol Endod. 1995 November; 80(5):540-54. Review
36. Almog D M, Benson B W, Wolfgang L, Frederiksen N L, Brooks S L. Computerized tomography-based imaging and surgical guidance in oral implantology. J Oral Implantol. 2006; 32(1):14-8
37. Harvey P D, Keefe R S. Studies of cognitive change in patients with schizophrenia following novel antipsychotic treatment. Am J Psychiatry. 2001 February; 158(2):176-84.
38. Schwartz C E, Bode R, Repucci N, Becker J, Sprangers M A, Fayers P M. The clinical significance of adaptation to changing health: a meta-analysis of response shift. Qual Life Res. 2006 November; 15(9):1533-50.

We claim the following:

1. A method for treating an animal or human subject suffering from a viral infection, the method comprising the steps of:
a. Selecting an agent, wherein the agent consists of a green tea extract, quercetin, licorice extract, cinnamomum extract, and selenium;
b. Administering the agent to the subject to decrease a symptom in said subject.

2. The method in claim 1, wherein said decrease a symptom of a viral infection, is a decrease in either the severity, the duration, or the frequency of said symptom, or any combination thereof.

3. The method in claim 1, wherein said symptom of a viral infection includes the standard signs and symptoms reported in the literature.

4. A method for treating an animal or human subject suffering from a viral infection, the method comprising the steps of:
a. Selecting an agent, wherein the agent consists of a green tea extract, quercetin, licorice extract, cinnamomum extract, and selenium;
b. Administering the agent to the subject to increase the general health of said subject.

5. The method in claim 1, wherein said virus is selected from the group consisting of Epstein Barr Virus (EBV), Cytomegalovirus (CMV), Herpes Simplex Virus 1 (HSV-1), Herpes Simplex Virus 2 (HSV-2), Human Papillomavirus (HPV), and Hepatitis C Virus (HCV).

6. The method in claim 2, wherein said virus is selected from the group consisting of Epstein Barr Virus (EBV), Cytomegalovirus (CMV), Herpes Simplex Virus 1 (HSV-1), Herpes Simplex Virus 2 (HSV-2), Human Papillomavirus (HPV), and Hepatitis C Virus (HCV).

7. The method in claim 3, wherein said virus is selected from the group consisting of Epstein Barr Virus (EBV), Cytomegalovirus (CMV), Herpes Simplex Virus 1 (HSV-1), Herpes Simplex Virus 2 (HSV-2), Human Papillomavirus (HPV), and Hepatitis C Virus (HCV).

8. The method in claim 4, wherein said virus is selected from the group consisting of Epstein Barr Virus (EBV), Cytomegalovirus (CMV), Herpes Simplex Virus 1 (HSV-1), Herpes Simplex Virus 2 (HSV-2), Human Papillomavirus (HPV), and Hepatitis C Virus (HCV).
